(19) United States
(12) Plant Patent Application Publication
(10) Pub. No.: US 2003/0172417 P1
(43) Pub. Date: Sep. 11, 2003

(54) Hybrid tea rose plant named 'Ruirorap'

(76) Inventor: Anton Pouw, De Kwakel (NL)

Correspondence Address: CHRISTIE, PARKER & HALE, LLP, 350 WEST COLORADO BOULEVARD, SUITE 500, PASADENA, CA, 91105, US

(21) Appl. No.: 10/095,931

(22) Filed: Mar. 11, 2002

(51) Int. Cl. A01H 5/00

(52) U.S. Cl. PLT/140

(57) ABSTRACT

A new and distinct Hybrid Tea Rose plant having attractive large Red flowers.

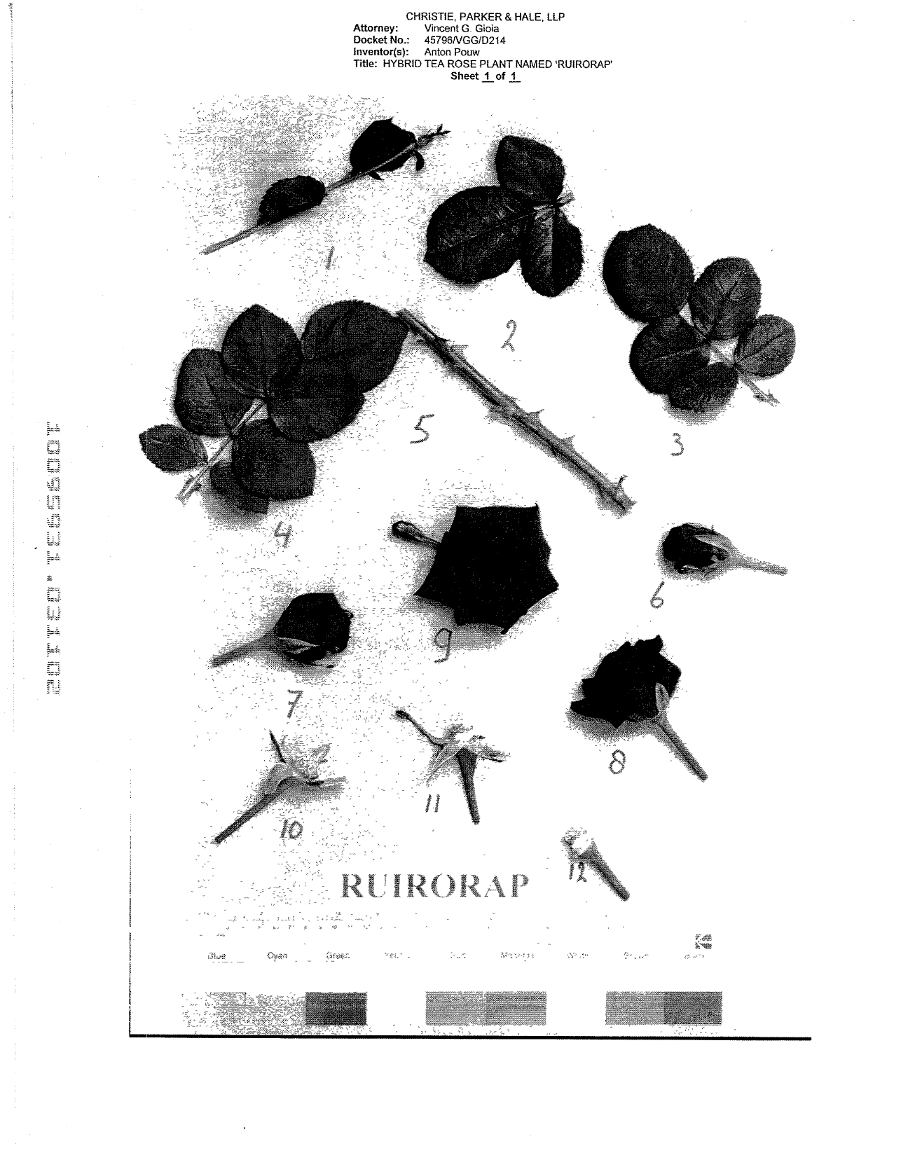

### BACKGROUND OF THE INVENTION

The present invention relates to a new and distinct variety of rose plant of the Hybrid Tea rose class, which was created by crossing as seed parent an unnamed, undistributed seedling and as a pollen parent the variety known as ‘Tina’. The varietal denomination of this new rose is named ‘RUIRORAP.’ The new variety was grown from a grafted cutting in The Netherlands.

### SUMMARY OF THE INVENTION

Among the novel characteristics possessed by this new variety which distinguishes it from its parents and all other varieties of which I am aware are its bud shape and color.

Asexual reproduction by propagation of grafting of the new variety as performed in De Kwakel, The Netherlands shows that the foregoing and other distinguishing characteristics come true to form and are established and transmitted through succeeding propagation.

### COMPARISON WITH PARENTS

Among other respects, the new variety differs from its parents in flower color, inasmuch as the seed parent has Pink flowers and the pollen parent has small Yellow flowers whereas the new variety has large Red flowers.

### BRIEF DESCRIPTION OF ILLUSTRATION(S)

The accompanying illustration shows a typical specimen of the new variety depicted in color as nearly true as is reasonably possible to make the same in a color illustration of this character.

### DESCRIPTION OF THE NEW VARIETY

The following is a detailed description of the new variety with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of 9-month old plants grown in a greenhouse in De Kwakel, The Netherlands during September from a grafted cutting.

### FLOWER

Blooming Habit: Continuous

Bud.—Large length about 5 cm, width about 3.5 cm Form: Cylindrical Color: When sepals first divide, Red darker than 46A.

Sepals.—At top some leafy protrusion, 2 sepals also have some leafy protrusion on the margins, surface looks lovely Size: Length about 4 cm, width at the base about 1 cm Color: Upper Surface: Near 144A Under surface: Near 144A and between 177A and 177B.

Peduncle.—Length: About 10 cm Aspect: First ca 2 cm have some small prickles about 1 mm long. At the top, the surface is smooth Strength: Diameter about 4 mm Color: Green Group, near 144A.

Bracts.—None observed.

Bloom

Size.—Average size when fully opened — about 12 cm.

Borne.—Singly.

Form.—Upper part of flower is flat (shape-side view), Lower part is convex (shape-side view).

Petalage: Number of petals under normal conditions — about 22

Color.—(⅓ to ½ open) Outer Petals: Top surface — Red Group, darker than 46A, under surface — Red Group near 46A Inner Petals: Top surface — Red Group darker than 46A, under surface — Red near 46A Red General tonality from a distance: Base of Petals (point of attachment): Very small White, near 155A.

Color (Fully Opened).—Outer Petals: Top surface — Red Group darker than 46A, under surface — Red Group near 46A Inner Petals: Top surface — Red Group darker than 46A, under surface — Red Group near 46A Red General tonality from a distance: Base of Petals (point of attachment): Very small White near 155A Color Change: Color does not change as bloom ages Red General tonality from a distance.

Texture.—Thick — wrinkled.

Appearance.—Inside velvety; outside satiny.

Form.—Nearly round, width about 6 cm, height about 6 cm.

Arrangement.—Irregularly arranged.

Petaloids in center.—About 5.

Persistence.—Petals are persistent.

Fragrance.—None.

Lasting quality.—On the plant: About 20 days In the vase: About 13 days, flower opens very slowly.

### REPRODUCTIVE ORGANS

Stamens, Filaments and Anthers

Arrangement.—Regularly arranged around styles.

Color.—Stamens Red Group near 53B, Filaments Red Group near 53B, Anthers Greyed-Yellow Group near 161B.

Pollen.—Color Greyed-White Group, near 156D.

Styles.—Bunched, uneven length.

Stigmas.—Color Grey-Yellow Group, near 161D.

Hips.—Shape: Pitcher shaped Size: At full bloom, length about 1 cm at top, width about 1 cm at top Color: This variety is not used as a mother plant.

### PLANT

Form.—Upright, long.

Growth.—Uniform branching, very vigorous at moderate temperatures; height attained (first season) about 180 cm.

Foliage.—Compound, 3, 5 to 7 leaflets Size: Length about 15 cm, width about 11 cm Quantity: About 11 leaves per stem, about 200 stems per m² per year Color New foliage: Upper side Purple Group near 187A; Under side Reddish Green Group, near 183A and 138B Mature foliage: Upper side Green Group near 139A; Under side Green Group near 137C Shape: Elliptic Texture Upper side: Smooth with deep-lying veins Under side: Smooth with high-lying veins Edge: Serrated Serration: Serrate.

Petiole.—Top surface Purple Group near 187A; Under surface near 138.

Stipules.—Length about 2 cm, margin serrated, color middle: Light Green between 138B to 138D; margin, Dark Green near 139A.

Auricle.—Absent.

Rachis.—1 to 3 prickles.

Resistance to disease.—Susceptible to Blackspot, resistant to Mildew and Rust.

### WOOD

New shoots

Bark.—Smooth, Reddish Green Group near 177A and 146B.

Wood.—Light Green Group Near 146B.

Old wood:

Bark.—Smooth, Green Group near 1346.

Wood.—White Grey Group near 155C.

Flower stem: Between 70 to 100 cm from last cut (including peduncle)

Thorns

Quantity.—Many small thorns (about 30 per stem) on basal canes at the basis, about 25 thorns per stem on laterals from basal canes.

Form.—Strong, curved downwardly, length about 1 cm, width about 0.5 cm, color (when young) Green near 146C, position irregular.

Prickles.—Some prickles at the basis of basal canes.

Winter Hardiness: Not tested — glasshouse variety

Uniqueness: Very good grow at moderate temperatures under glasshouse conditions, good bud shape and color

I claim:

1. A new and distinct Hybrid Tea Rose plant of the variety substantially as shown and described.
